(19) United States
(12) Patent Application Publication
(10) Pub. No.: US 2011/0213276 A1
(43) Pub. Date: Sep. 1, 2011

(54) APPARATUS AND METHOD FOR MONITORING ACTIVITY LEVEL

(76) Inventors: Israel SARUSSI, (US); Michael Braunold, (US)

(21) Appl. No.: 13/029,420

(22) Filed: Feb. 17, 2011

Related U.S. Application Data
(60) Provisional application No. 61/305,571, filed on Feb. 18, 2010.

(51) Int. Cl. A61B 5/11

(52) U.S. Cl. 600/595

(57) ABSTRACT

A device and a method for monitoring a person's movement and activity levels is provided. The device includes a microcontroller, an accelerometer and a visual indicator in communication with the microcontroller. The accelerometer is configured to continually check a person's movement in at least two of three axes being measured and to display the person's wellbeing based on the person's level of activity over a pre-defined period of time.

### CROSS REFERENCE TO RELATED APPLICATIONS

The present application relates to and claims priority from a provisional application entitled “An Apparatus and a Method for Monitoring a Person's Movement and Activity Levels” filed Feb. 18, 2010 and assigned Ser. No. 61/305,571, describing and illustrating a related invention made by the present inventor.

### FIELD OF THE INVENTION

The present invention relates to an apparatus and a method for monitoring a person's movement and activity levels.

### BACKGROUND OF THE INVENTION

One of the major problems affecting today's generation is the problem of obesity. Approximately, twenty percent of the population in the United States suffers from being overweight.

Obesity has been defined by the National Institutes of Health as a BMI (body mass index), of 30 and above. (A BMI of 30 is about 30 pounds overweight.)

Overweight is a significant contributor to health problems. It increases the risk of developing a number of diseases including Type 2 (adult-onset) diabetes, High blood pressure (hypertension), strokes, heart attacks and heart failure, for example.

Though obesity is a problem amongst all socio-economic levels, a significant proportion of the population which is overweight belong to the lower socio-economic levels, and generally have a lower health awareness and do not use gyms and fitness centers. The lower socio-economic levels will not spend money on sport watches, which are priced to appeal to the higher socio-economic levels.

It is appreciated, especially by the medical profession that there is a need to reduce weight of obese patients for the general health and wellness. It is also accepted that one's health may be improved by regular daily activity, such as walking a minimum of recommended steps.

There is thus a need for an apparatus and method capable of monitoring and transmitting the physiological and wellness parameters. Thus, it would be advantageous to be able to measure and monitor the general wellness of a person on a daily basis and to display a visual indication of his activity during the time.

### SUMMARY OF THE INVENTION

The present invention describes embodiments of a method and system for accurately determining the heart pulse rate in situations where the signal being measured is likely to be affected by ‘noise’ and other disturbances.

There is therefore provided, in accordance with an embodiment of the present invention a device and a method for monitoring a person's movement and activity levels. The device includes a microcontroller, an accelerometer and a visual indicator in communication with the microcontroller. The accelerometer is configured to continually check a person's movement in at least two of three axes being measured and to display the person's wellbeing based on the person's level of activity over a pre-defined period of time.

Furthermore, in accordance with an embodiment of the present invention, the visual indicator includes a plurality of differently colored LEDs (light emitting diodes), in communication with the microcontroller, wherein each of plurality of differently colored LEDs represents a different level of activity.

Furthermore, in accordance with an embodiment of the present invention, the device includes at least one counter in communication with the microcontroller and the counters are configured to record any of a group of activities including the number of steps or movement made by the user over a pre-defined period of time.

Furthermore, in accordance with an embodiment of the present invention, a first counter is configured to record activity over a period of one hour and a second counter is configured to record activity over a period of a 24 hourly (one day) period of time.

Furthermore, in accordance with an embodiment of the present invention, the device further includes a blood saturation and heat pulse rate monitor in communication with the microcontroller. The visual indicator is configured to display an indication of the wellbeing of the user of the device based upon the recorded blood saturation level and/or heart pulse rate.

In addition, there is provided, in accordance with an embodiment of the present invention a method for monitoring a person's movement and activity levels, which includes measuring a person's movement in three axes during a pre-defined period of time and visually indicating a person's wellbeing based on the person's level of activity in least two of three dimensions measured over the pre-defined period of time.

Furthermore, in accordance with an embodiment of the present invention, the method further includes the step of activating one of a plurality of differently colored LEDs (light emitting diodes), wherein each of said plurality of differently colored LEDs represents a different level of activity.

Furthermore, in accordance with an embodiment of the present invention, the method further includes the step of: if movement has occurred in least two of three dimensions, advancing a first counter for the pre-defined period of time by one and if the pre-defined period of time is one hour and the hourly time period has not changed, advancing a second counter for a 24 hourly period by one.

Furthermore, in accordance with an embodiment of the present invention, the step of activating includes the person being monitored activating a control to illuminate the respective colored LED indicating the person's current wellbeing status.

Furthermore, in accordance with an embodiment of the present invention, the method further includes the step of: if the pre-defined period of time is one hour and the hourly time period has advanced to the next hourly time period, advancing the second counter for a 24 hourly period by one and reducing the corresponding counter movements from the previous day's movement from the second counter.

Furthermore, in accordance with an embodiment of the present invention, the method further includes the step of: measuring the blood saturation and heat pulse rate monitor of the person; and indicating the wellbeing of the user of the device based upon at least one of the recorded blood saturation level and the heart pulse rate.

### BRIEF DESCRIPTION OF THE DRAWINGS

The present invention will be understood and appreciated more fully from the following detailed description taken in conjunction with the appended drawings in which:

FIG. 1 is a perspective view of a bracelet device for monitoring a person's activity level in accordance with an embodiment of the invention;

FIG. 2 is a schematic block diagram illustration of the components comprising the bracelet device of FIG. 1; and

FIG. 3 is a schematic flow chart illustration of the method for monitoring a person's activity level in accordance with an embodiment of the invention.

### DESCRIPTION OF THE INVENTION

The present invention relates to a device and a method for monitoring a person's movement and activity levels.

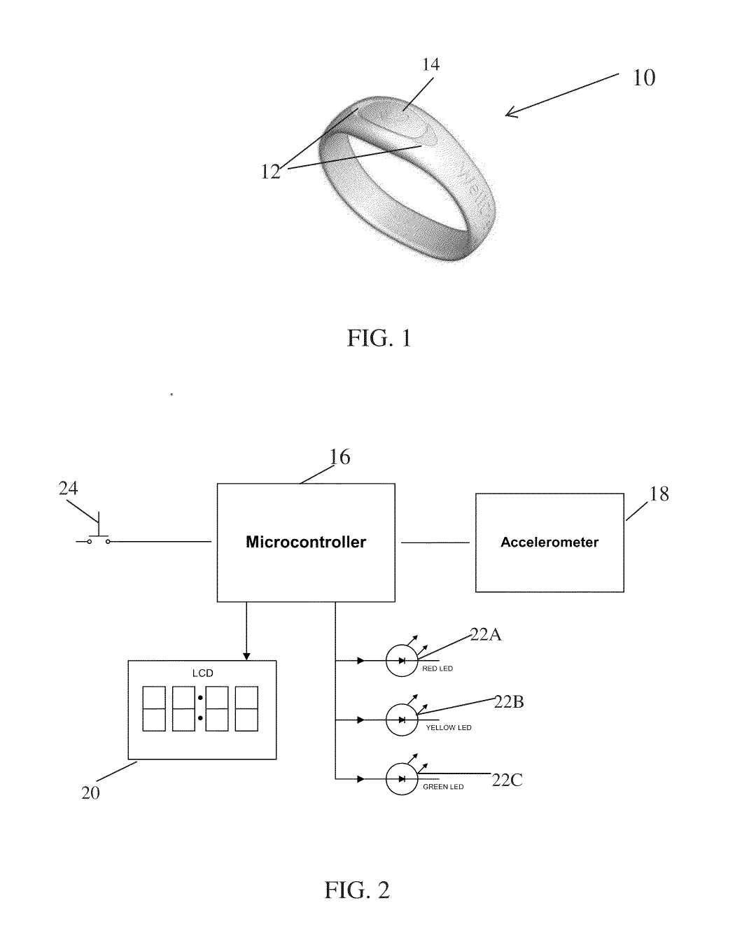

FIG. 1, to which reference is now made, is a perspective view of a device in the shape of a bracelet, generally designated 10, for monitoring a person's activity level in accordance with an embodiment of the invention.

The bracelet is an exemplary illustration of a device, which may be used for monitoring and visually indicating a person's activity or movement during the day, for example. As will be appreciated by persons knowledgeable in the art, the device is not restricted to shape of a bracelet, but may be configured in any suitable shape such as a watch, a pin or any other accessory, for example, which may be attached to the hand, leg, belt or body of the user.

Motion detectors in the form of pedometers are common in today's market. Unfortunately, their use is limited to the population with high health awareness. Furthermore, the user is required to have a minimum amount of knowledge regarding the recommended physical activity and generally may need to actively input data which at the very least is annoying and also the user is required to reset the pedometer from time to time.

The device of the present invention has being designed especially for persons who don't want to be enslaved to a device, which requires any form of input such as making calculations or even the need to reset it. The device 10 comprises a visually indicator 12, which indicates the activity level of the wearer.

In one embodiment, the visually indicator 12 may be a series of pre-defined colors. While each color represents a different level of activity. For example, red may indicate ‘lazy’ activity (or relative inactivity), yellow for medium activity and green for reasonable activity. The data may be stored by the FIFO (first in, first out) principle, gathering current data and deleting the data over a pre-determined time period, such as 24 hours, for example. The device 10 may also comprise a display button 14, which may optionally be activated to display the current activity level of the user.

The device may be configured to keep a count of the number of steps or movement made by the user. This data is kept in running memory, for the pre-determined time period. By pressing the button 14, the relevant color light will be displayed, indicating to the user his activity level for the time period.

The device may also be configured to incorporate a watch or other time piece. Furthermore, the device may be configured as a wrist watch and incorporate components for the non-invasive measurement of blood saturation and heart pulse rate to display the well-being of the wearer, for example, as described in U.S. Pat. No. 7,613,490, assigned to the assignees of the present application.

The device 10 may be configured to utilize known in the art pedometer technology. Reference is now made to FIG. 2, which is a schematic block diagram illustration of illustration of the components inside the device 10. The device comprises a microcontroller 18 in communication with an accelerometer 18, LCD display 20 and a plurality of differently colored LEDs 22a, 22b and 22c. Power may be provided via a battery 24.

The accelerometer 16, or similar device, measures acceleration in three dimensions. Within the pre-defined cycle (say a day of 24 hours), the movements of the bracelet is tracked. Whenever movement of a pre-defined intensity occurs on at least two axes, a step is registered and the counting meter is advanced by one. Movement is thus recorded for a plurality of time slots (each hour over a period of 24 hours, for example). After 24 hours, the number of movements (steps) of the first hour are deleted and the number of steps of the next hour are added in, on the FIFO principle.

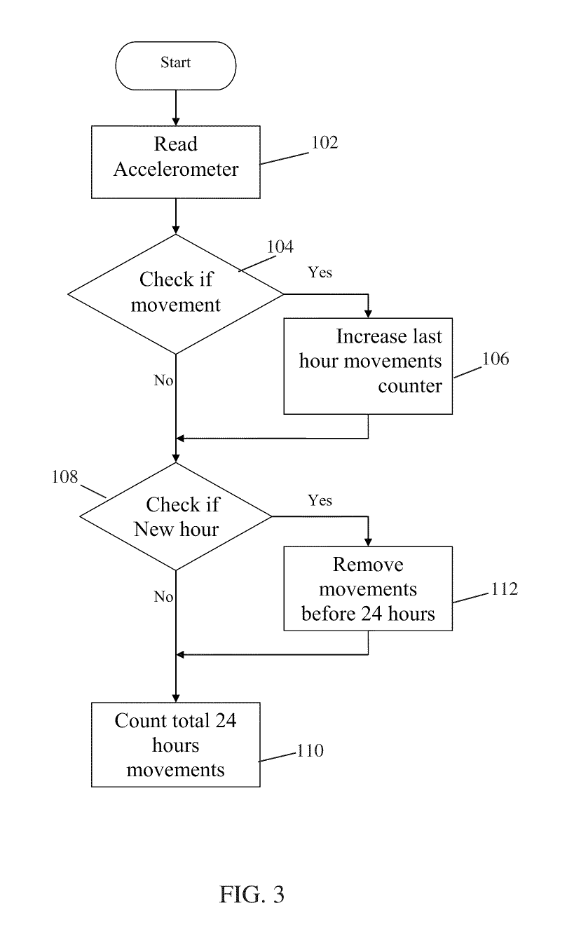

Reference is now made to FIG. 3, which is a schematic flow chart illustration of the method for monitoring a person's activity level in accordance with an embodiment of the invention. For exemplary purposes only, 24 hourly periods are used. It will be appreciated that any other period, such as 48 hours, for example, may be used

The accelerometer 18 is read (step 102) and a check for movement in at least two axes is made (query box 104). If movement has occurred, the counter for that hourly period is advanced (step 106). A check of the time is made (query box 108). If the hourly time slot has not changed, the total for the 24 hour period is also advanced (step 110). If the hourly time slot has changed (say, the tine has moved to 3:01 and the hourly slot is now 3 pm to 4 pm), the number of movements from the corresponding time slot (3 pm to 4 pm) from the previous day is removed from the day's total (step 112).

Thus, whenever the wearer, activates the button 14, one of the colored LEDs will be illuminated. The color will be determined by the number of steps taken by the wearer during the last 24 hours. For example, more than 10,000 steps-which would indicate good intensive physical activity will activate a green light. Medium activity (say 6000 steps) would be indicated by a yellow light while a red light would be indicative of little or no physical activity.

The foregoing description of the embodiments of the invention has been presented for the purposes of illustration and description. It is not intended to be exhaustive or to limit the invention to the precise form disclosed. It should be appreciated by persons skilled in the art that many modifications, variations, substitutions, changes, and equivalents are possible in light of the above teaching. It is, therefore, to be understood that the appended claims are intended to cover all such modifications and changes as fall within the true spirit of the invention.

1. A device for monitoring a person's movement and activity levels, comprising:
a microcontroller;
an accelerometer in communication with the microcontroller;
a visual indicator in communication with the microcontroller;
wherein said accelerometer is configured to check for movement in at least two axes; and
wherein the level of activity is displayed by the visual indicator.

2. The device according to claim 1, wherein said visual indicator comprises:
a plurality of differently colored LEDs (light emitting diodes), in communication with the microcontroller,
wherein each of plurality of differently colored LEDs represents a different level of activity.

3. The device according to claim 1, further comprising:
at least one counter in communication with the microcontroller;
wherein said at least one counter is configured to record any of a group of activities including the number of steps or movement made by the user over a pre-defined period of time.

4. The device according to claim 1, wherein said at least one counter comprises a counter configured to record activity over a period of one hour and a counter configured to record activity over a period of a 24 hourly (one day) period of time.

5. The device according to claim 1 further comprising:
a blood saturation and heat pulse rate monitor in communication with the microcontroller,
wherein said visual indicator is configured to display an indication of the wellbeing of the user of the device based upon at least one of the recorded blood saturation level and the heart pulse rate.

6. A method for monitoring a person's movement and activity levels, comprising:
measuring a person's movement in three dimensions during a pre-defined period of time; and
visually indicating a person's wellbeing based on the person's level of activity in least two of three dimensions measured over the pre-defined period of time.

7. The method according to claim 6, further comprising the step of:
activating one of a plurality of differently colored LEDs (light emitting diodes), wherein each of said plurality of differently colored LEDs represents a different level of activity.

8. The method according to claim 7, further comprising the steps of:
the step of activating comprises the person being monitored, activating a control to illuminate the respective colored LED indicating the person's current wellbeing status.

9. The method according to claim 6, further comprising the steps of:
if movement has occurred in least two of three dimensions, advancing a first counter for the pre-defined period of time by one; and
if the pre-defined period of time is one hour and the hourly time period has not changed, advancing a second counter for a 24 hourly period is advanced by one.

10. The method according to claim 6, further comprising the steps of:
if the pre-defined period of time is one hour and the hourly time period has advanced to the next hourly time period, advancing the second counter for a 24 hourly period by one and reducing the corresponding counter movements from the previous day's movement from the second counter.

11. The method according to claim 6, further comprising the steps of:
measuring the blood saturation and heat pulse rate monitor of the person; and
indicating the wellbeing of the user of the device based upon at least one of the recorded blood saturation level and the heart pulse rate.
